(19) United States
(12) Patent Application Publication
(10) Pub. No.: US 2020/0022620 A1
(43) Pub. Date: Jan. 23, 2020

(54) Base Mount Apparatus and Method of Using Same

(71) Applicant: Kinesics, LLC, Baton Rouge, LA (US)

(72) Inventor: Gerald Drefahl, Baton Rouge, LA (US)

(21) Appl. No.: 16/515,228

(22) Filed: Jul. 18, 2019

Related U.S. Application Data
(60) Provisional application No. 62/700,528, filed on Jul. 19, 2018.

(51) Int. Cl. A61B 5/107; A61B 5/00; A61B 5/11

(52) U.S. Cl. CPC A61B 5/1071; A61B 5/1121; A61B 5/4528

(57) ABSTRACT

A base mount apparatus for use in attachably connecting to a range of motion measurement device, such as, an electronic goniometer, particularly, for example, a digital HALO medical device. More particularly, the present invention comprises a base mount apparatus having a substantially planar surface and at least one substantially planar and flat edge that is attachably connected to a range of motion measurement device for use in increasing a variety of different capabilities of said range of motion measurement device.

### CROSS REFERENCES TO RELATED APPLICATION

Priority of U.S. Provisional Patent Application Ser. No. 62/700,528, filed Jul. 19, 2018, incorporated herein by reference, is hereby claimed.

### STATEMENTS AS TO THE RIGHTS TO THE INVENTION MADE UNDER FEDERALLY SPONSORED RESEARCH AND DEVELOPMENT

None

### BACKGROUND OF THE INVENTION

### Field of the Invention

The present invention pertains to a base mount apparatus for use in attachably connecting to a range of motion measurement device, such as, an electronic goniometer, particularly, for example, a digital HALO medical device. More particularly, the present invention pertains to a base mount apparatus that is attachably connected to a range of motion measurement device for use in increasing a variety of different capabilities of said range of motion measurement device.

### Brief Description of the Prior Art

In typical medical professional offices, an assessment of the human body is a critical tool in identifying any imbalances, restrictions in mobility and flexibilities, and asymmetries within the human body, as well as measuring any improvements in mobility and flexibility. These assessments of the body are typically done by traditional measuring devices, such as, for example, a goniometer. A goniometer is an instrument that is used for the precise measurement of angles, that is typically used to measure a range of motion around a joint in the body, and that is typically used in a physical therapy or other similar medical setting. For example, a HALO medical device, is a specific type of digital goniometer that utilizes substantially long lasers in order to intersect landmarks on a person's body for a repeatable measure.

Although these traditional measuring devices are conventionally used, these conventional goniometers are generally manual and/or analog, which can cause inefficiencies, are subject to error, and lack repeatability during measurements. As such, there is a need for a device that can assist a digital goniometer, or any other similar range of motion measurement device, in order to enhance proficiency, efficiency, and repeatability.

### SUMMARY OF THE INVENTION

The present invention, in conjunction with a digital measurement device, such as, for example, a HALO medical device, or any other similar electronic range of motion measurement devices, is designed to increase functionality, stability, and accuracy, and enhance proficiency, competency, efficiency, and repeatability in measurements. The HALO digital measurement device, and any other similar electronic measurement devices or goniometers, typically incorporates lasers for alignment and an accelerometer for measurement. When the HALO device is used as a standalone product, there are several deficiencies present, such as, for example, a decreased ability to ensure laser alignment, and being inadvertently tiled due to a substantially round shape of the device, which can cause error in measurement.

Conversely, when the HALO device is mounted to the base mount apparatus of the present invention, the features of the base mount allow for an increase in the number of possible measurements, and thus, standardizes the process by which these measurements are collected, thereby allowing for a more consistent result. Furthermore, due to a variety of features of the base mount apparatus of the present invention that allow for greater levels of stabilization and a more efficient placement amongst various anatomical landmarks and areas of the human body, the overall measurement process is accelerated and thus more effective.

The base mount apparatus of the present invention can be utilized during three categories of principles for assessing kinematic movement and determining levels of flexibility and mobility within both a single and multiple joint segments: Arthrokinematic—Joint mobility (appendicular); Osteokinematic—Segmental spinal mobility (axial); and, Myokinematic—Muscular flexibility.

An arthrokinematic assessment generally gives the professional guidance to determine precise corrective flexibility and mobility programs to allow for successful execution of exercises and integrated movement patterns. By way of illustration, but not limitation, a program that is designed for arthrokinematic assessment typically allows for the following:
Giving professional guidance to foundational and functional indications (what should be done), contraindications (what shouldn't be done), modifications (what to modify so that it can be done), and paths to progression for individual exercises and overall programs to reduce injury rates and increase motivational strategies;
Providing detailed analysis of appendicular musculoskeletal and neuromuscular joint restrictions and their associated levels of muscular flexibility;
Allowing for comparative analysis of asymmetries between right and left sides of the body;
Associating possible muscular imbalances with postural deviations;
Clarifying movement systems and fascial trains areas of isolated tension and restrictions that lead to integrated compensations and dysfunctions;
Influencing variables in strength and performance training such as equipment selection, intensity, speed, incline and load selection;
Providing a communication tool to the participant as to their current level of health and fitness, and giving insight into associated contributing factors to areas of pain and/or dysfunction(s);
Serving as a motivational tool allowing the professional to develop definitive measurable and quantifiable goals for each of the participant's objectives; and
Improving accountability, for the participant and professional, by establishing a baseline and allowing for continual re-evaluation of progress towards determined goals.

Thus, an arthrokinematic assessment (AKA) allows for an extensive joint by joint assessment of a body's range of motion to determine isolated muscle-length tension imbalances that are used to formulate a corrective program for restoring flexibility and mobility to the entire body. The total time that is typically needed for this particular assessment will be approximately thirty (30) minutes.

An osteokinematic assessment generally allows for the following:
Providing a detailed analysis of axial musculoskeletal and neuromuscular restrictions with their associated levels of segmental mobilities;
Defining head, torso, spinal, and pelvic angles as they relate to optimal postural alignment and function;
Allowing for comparative analysis of asymmetries between right and left sides of the body;
Associating osteokinematic assessment spinal angles, immobilities, and asymmetries with postural deviations in a bioprint report;
Clarifying the movement systems, anatomy trains and integrated lines of tension, along with their associated implications on kinetic joint functions;
Providing a communication tool to the participant as to their current level of health, fitness, and function and gives insight into associated contributing factors to areas of pain and/or dysfunction(s);
Serving as a motivational tool allowing the professional to develop definitive measureable and quantifiable goals for each of the participant's objectives;
Giving the professional guidance to determine precise corrective flexibility and mobility programs to allow for successful execution of exercises and integrated movement patterns;
Allowing the professional to choose the appropriate corrective exercise: such as, indications (what should be done), contraindications (what shouldn't be done), modifications (what to modify so that it can be done), and paths to progression for individual exercises and overall programs to reduce injury rates and increase motivational strategies; and
Establishing a baseline and continual feedback on programs efficacy and effectiveness via re-assessments and re-evaluations.

Thus, an orthokinematic assessment (OKA) is similar to an AKA, and typically provides an in depth assessment of the alignment, position, and range of motion of a skeletal system (spine, pelvis, and other essential anatomical points) so that any changes in a body's structure and function can be effectively monitored. The total time that is typically needed for this particular assessment will be approximately twenty (20) minutes.

### BRIEF DESCRIPTION OF THE DRAWINGS/FIGURES

The foregoing summary, as well as any detailed description of the preferred embodiments, is better understood when read in conjunction with the drawings and figures contained herein. For the purpose of illustrating the invention, the drawings and figures show certain preferred embodiments. It is understood, however, that the invention is not limited to the specific methods and devices disclosed in such drawings or figures.

FIG. 1 depicts FIG. 1 depicts a side view of a preferred embodiment of a base mount apparatus of the present invention illustrating a substantially flat edge placement.

FIG. 2 depicts a side view of a preferred embodiment of a base mount apparatus of the present invention illustrating another substantially flat edge placement.

FIG. 3 depicts a side view of a preferred embodiment of a base mount apparatus of the present invention illustrating a substantially flat surface placement.

FIG. 4 depicts a side view of a preferred embodiment of a base mount apparatus of the present invention illustrating an enhanced view of a substantially flat surface placement.

FIG. 5 depicts a side perspective view of a preferred embodiment of a base mount apparatus of the present invention with a HALO device that has been laser aligned on a plurality of alignment crosshairs of said base mount apparatus.

FIG. 6 depicts a side view of a preferred embodiment of a base mount apparatus of the present invention illustrating a method of prong usage.

FIG. 7 depicts a side view of a preferred embodiment of a base mount apparatus of the present invention illustrating an alternate method of prong usage.

FIG. 8 depicts a side view of a preferred embodiment of a base mount apparatus of the present invention illustrating an enhanced view of a method of prong usage.

FIG. 9A depicts a front view of a preferred embodiment of a base mount apparatus of the present invention.

FIG. 9B depicts a side view of a preferred embodiment of a base mount apparatus of the present invention

FIG. 10A depicts a front view of a preferred embodiment of a base mount apparatus of the present invention.

FIG. 10B depicts a rear view of a preferred embodiment of a base mount apparatus of the present invention.

FIG. 11A depicts a front view of a preferred embodiment of a base mount apparatus of the present invention.

FIG. 11B depicts a rear view of a preferred embodiment of a base mount apparatus of the present invention.

FIG. 12A depicts a front view of a preferred embodiment of a base mount apparatus of the present invention.

FIG. 12B depicts a rear view of a preferred embodiment of a base mount apparatus of the present invention.

Dimensions and material selections are illustrative only and are not, and should not be, construed as limiting in any way.

### DETAILED DESCRIPTION OF A PREFERRED EMBODIMENT

Referring to the drawings, the present invention comprises a base mount apparatus 10 that can be manufactured from a substantially rigid material, such as, for example, a plastic or acrylic material, or any other material exhibiting like characteristics. The base mount apparatus 10 of the present invention comprises a substantially planar surface that is used in order to be positioned on a person's body in an accurate manner and increase a number of range of motion measurements. By way of illustration, but not limitation, base mount apparatus comprises a substantially square or rectangular shape having four (4) sides; however, it is to be observed that said surface of base mount apparatus 10 can be manufactured in a variety of different shapes and/or sizes depending on a particular user's needs.

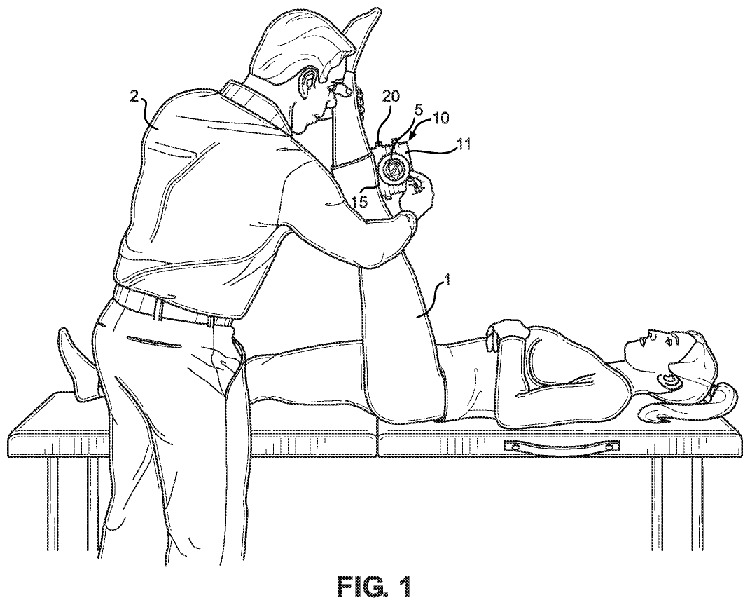

Referring to FIGS. 1 through 8, the present invention comprises base mount apparatus 10 that is used in order to be positioned on a person's body 1 accurately and thus increase a number of range of motion measurements from said user 1. Base mount apparatus 10 is able to beneficially mount a digital or electronic range of motion measurement device 5, such as, for example, a HALO medical device, in order to take more accurate measurements of a person's body 1, thereby increasing functionality, stability, accuracy, proficiency, and efficiency of said measurements. Said HALO medical device 5 is continuously referred to herein and depicted in the figures herein as the digital range of motion measurement device that is attachably connected to base mount apparatus 10; however, it is to be observed that any other similar range of motion measurement device can alternatively be used instead of HALO medical device 5.

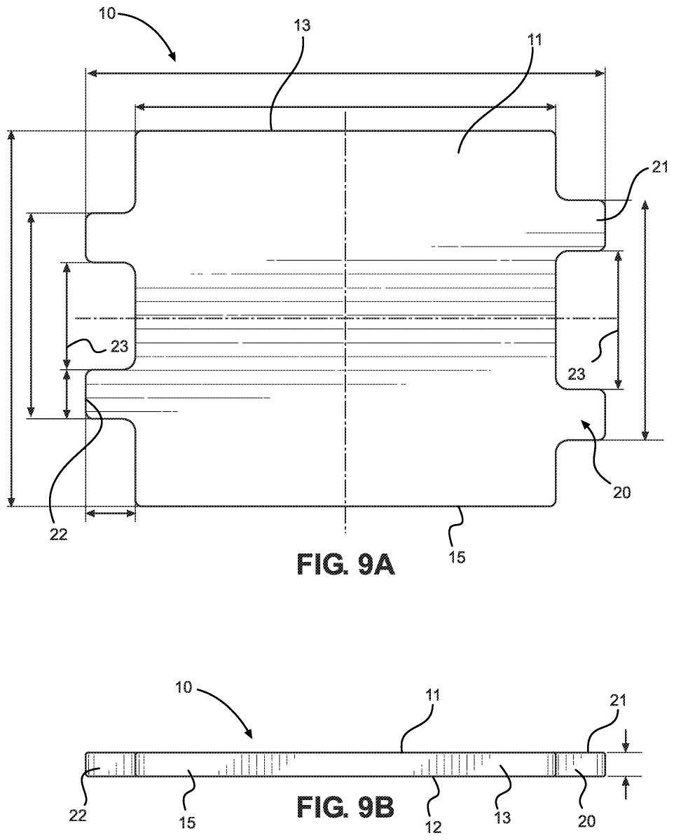

FIG. 9A depicts an aerial view of a preferred embodiment of a top surface 11 of said base mount apparatus 10 of the present invention. FIG. 9B depicts a side view of a preferred embodiment of said base mount apparatus 10 of the present invention. Referring to FIGS. 9A and 9B, in a preferred embodiment, said base mount apparatus 10 of the present invention generally comprises a substantially planar surface, having a top surface 11 and a bottom surface 12, and a plurality of sides 13, wherein at least one of sides 13 comprises a substantially flat and planar edge 15, and wherein at least another one of sides 13 comprises a set of prongs 20. Edge 15 is used in order to be positioned on a user's body accurately and increase a number of range of motion measurements. The number of flat edges 15 can be singular or can comprise multiple edges 15 in order to fit the body properly.

The substantially flat, planar shape of base mount apparatus 10 allows for a substantially flat surface and/or straight edge 15 placement on a user's body. Thus, edge 15 minimizes a variety of different changes for inadvertent rotation, decreases error in placement, and increases repeatability. The planar, flat shape spaces across certain body parts in a particular manner while having multiple points of contact, whereas a substantially smaller and rounder shaped device might not sit level, thus causing the device to rotate, thereby affecting the validity and reliability of the measurement that is being taken.

In a preferred embodiment, still referring to FIGS. 9A and 9B, base mount 10 further comprises a plurality of prongs 20—typically two (2) sets of alignment prongs (a first set 21 and a second set 22)—in a variety of different sizes and spacing that are used to position base mount 10 on the body accurately and increase a number of range of motion measurements, wherein each set of prongs comprises a first prong and a second prong. By way of illustration, but not limitation, first set of alignment prongs 21 can have substantially larger spacing 23 than second set of alignment prongs 22, wherein spacing 23 between said first set of prongs 21 can be approximately 1.5″ in length, and spacing 23 between said second set of prongs 22 can be approximately 1.25″ in length. Prong spacing 23 can comprise a variety of different geometries, including, but not limited to, a plurality of substantially flat, planar edges, a plurality of substantially rounded edges, or a plurality of substantially pointed vertices. In addition, said prongs 20 can also comprise a variety of different colors in order to assist a user in distinguishing proper measurements. Moreover, prongs 20 are used to align base 10 and a center of the base 10 accurately along parts of the person's body. For example, the substantially larger spacing 23 on first set of prongs 21 is to be used for substantially larger body parts and/or larger framed individuals. The prongs 20 also ensure a plurality—typically two (2)—points of contact with the body, which thus increases stability when obtaining particular measurements.

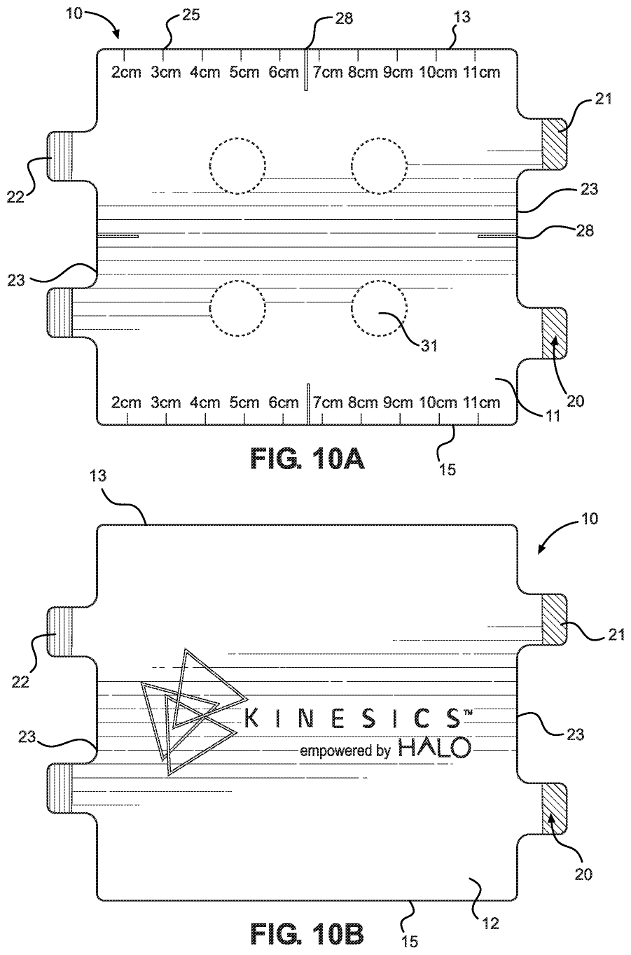

FIG. 10A depicts an aerial view of a preferred embodiment of top surface 11 of said base mount apparatus 10 of the present invention. FIG. 10B depicts a bottom view of a preferred embodiment of bottom surface 12 of said base mount apparatus 10 of the present invention. Referring to the drawings, FIGS. 10A and 10B depict base mount apparatus 10 of the present invention generally comprising a substantially planar surface, said prongs 20, and at least one ruler(s) 25 for appropriate measurement reading. Ruler 25 is typically positioned along a side edge 15 of base mount apparatus 10 in order to adjacently align to a person's body. Moreover, said ruler 25 can be used as an additional measurement device, not necessarily in conjunction with the HALO device 5, or any other digital range of motion measurement device.

Alternatively, ruler 25 can also be used to provide a different type of alignment of base mount apparatus 10 of the present invention and HALO device 5 with respect to each other and with respect to a particular body part. By way of illustration, but not limitation, if ruler 25 is ruled in degrees and shaped like a protractor or a compass, the HALO device 5 could be mounted in a variety of different ways. The HALO device 5 can be oriented to align with various geometries on base 10, thus being beneficial in capturing additional range of motion measurements.

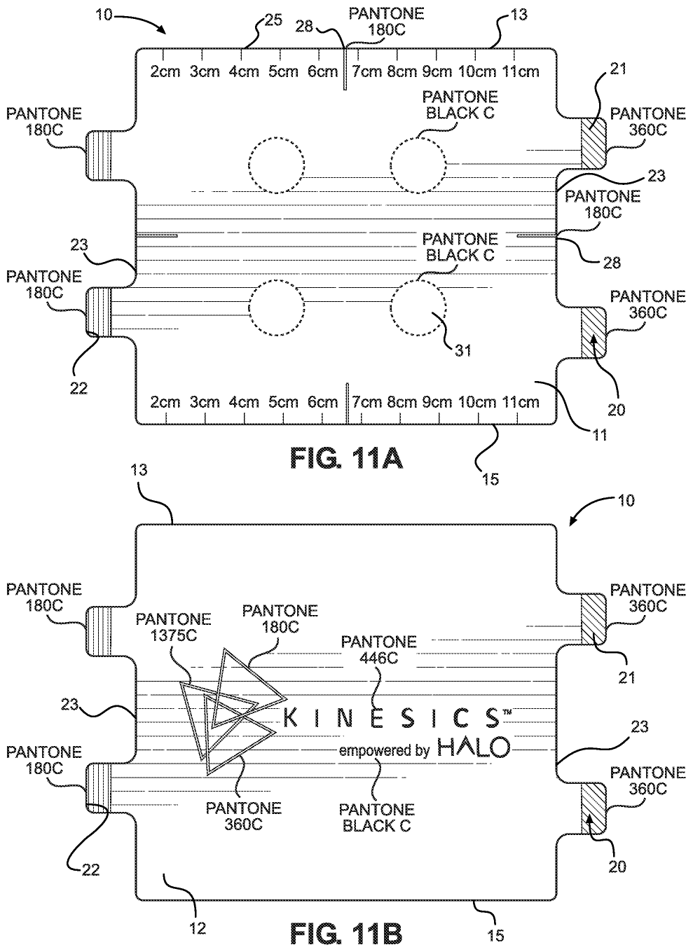

FIG. 11A depicts an aerial view of a preferred embodiment of base mount apparatus 10 of the present invention, while FIG. 11B depicts a bottom view of a preferred embodiment of base mount apparatus 10 of the present invention. Referring to FIGS. 11A and 11B, in a preferred embodiment, base mount apparatus 10 of the present invention generally comprises a plurality of crosshairs 28 located on top surface 11 of base mount apparatus 10. Said crosshairs 28 assist in accurately aligning said HALO device 5, thus allowing for precise orientation of base edges 15 of base mount apparatus 10 with the HALO device 5. Crosshairs 28 of base mount 10 of the present invention increases the accuracy of alignment by providing a landmark focal point that is near to the HALO device 5. Moreover, said crosshairs 28 also allow for laser alignment in conjunction with the specific geometry of base mount 10, thereby allowing for precise placement on different body parts. For example, prongs 20, along with crosshairs 28 and a laser from HALO device 5, can provide for accuracy and precision in body part bisection and measurement.

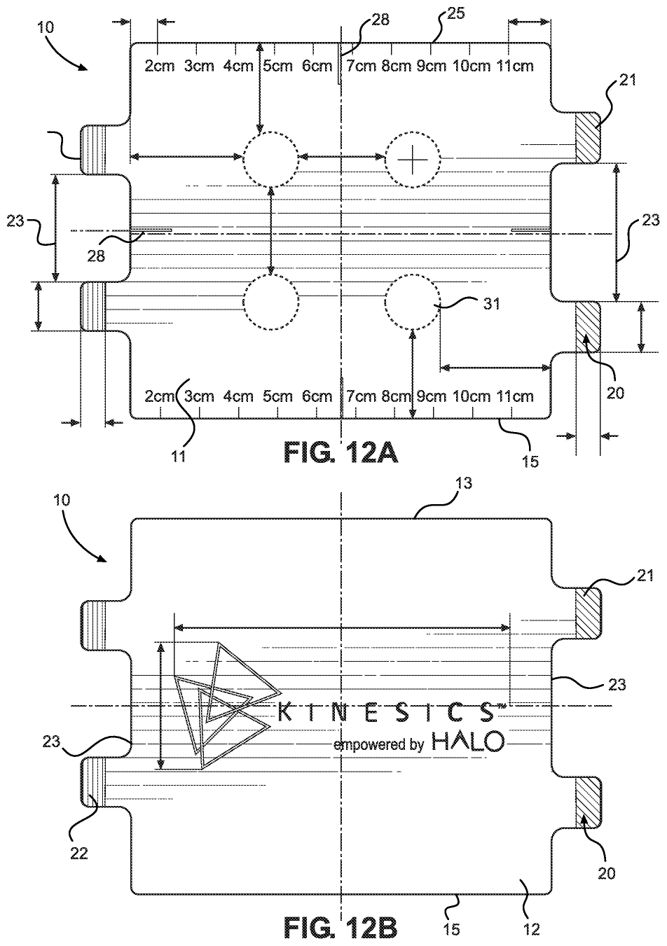

FIG. 12A depicts an aerial view of a preferred embodiment of a base mount apparatus 10 of the present invention, while FIG. 12B depicts a bottom view of a preferred embodiment of base mount apparatus 10 of the present invention. Referring to the drawings, FIGS. 12A and 12B depict base mount apparatus 10 of the present invention generally comprising a plurality of notches or grooves 31, wherein said notches 31 allow for said HALO device 5, or any other similar goniometer or digital range of motion measurement device, to adhere or attachably connect to base mount apparatus 10. Thus, base mount apparatus 10 comprises a method of connecting a HALO digital measurement device 5 to said base mount apparatus 10. Moreover, base mount 10 further comprises a method of aligning said HALO digital measurement device 5 to a plurality of geometry profiles, wherein said different geometry profiles that are used, such as a substantially long edge or a substantially short edge, can depend on the particular body part that is being measured.

By way of illustration, but not limitation, top surface 11 of base mount 10 comprises a hook and loop pad (such as, for example, Velcro®) located within said notches 31 of top surface 11 of base mount 10, or any other similar attachment means, that can be oriented on its axis or a cross pattern. Although not depicted in FIG. 12A or 12B, in conjunction with said hook and loop pad located on top surface 11 of base mount 10, HALO device 5 also comprises an opposing match to said hook and loop pad, thereby allowing HALO device 5 to attachably connect to top surface 11 of base mount 10. Additionally, although not illustrated in FIG. 12A or 12B, base 10 can alternatively incorporate an attachment means by way of a “snap in” or “slide in” connection or configuration, wherein no modifications to the HALO device 5 are necessary in order to connect said HALO device 5 to base mount apparatus 10.

Referring back to FIG. 1, FIG. 1 depicts a side view of a physical therapist 2 utilizing substantially flat edge placement of base mount apparatus 10 on a patient's leg 1. As such, physical therapist places flat edge 15 of base mount 10 adjacently along patient's leg 1, or any other body part in need of measuring. Physical therapist 2 is then able to use HALO device 5 in conjunction with base mount 10 in order to take a proper and accurate measurement of patient's body 1 in order to determine patient's particular range of motion.

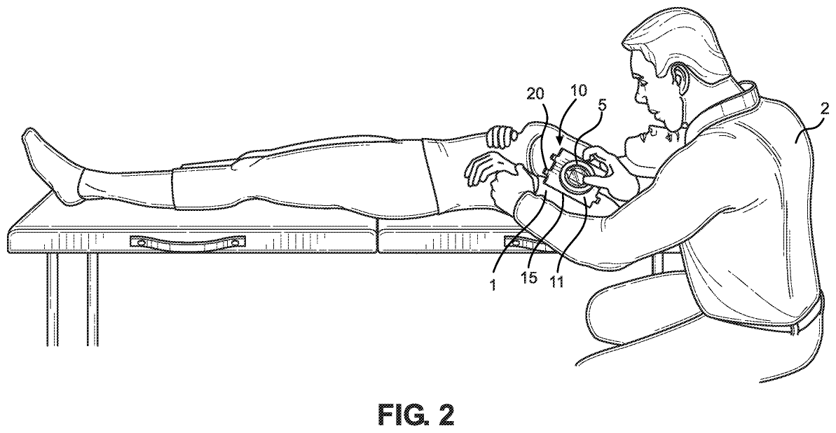

FIG. 2 depicts a side view of a physical therapist 2 utilizing substantially flat edge placement of base mount apparatus 10 on a patient's arm 1. As such, physical therapist places flat edge 15 of base mount 10 adjacently along patient's arm 1, or any other body part in need of measuring. Physical therapist 2 is then able to use HALO device 5 in conjunction with base mount 10 in order to take a proper and accurate measurement of patient's body 1 in order to determine patient's particular range of motion.

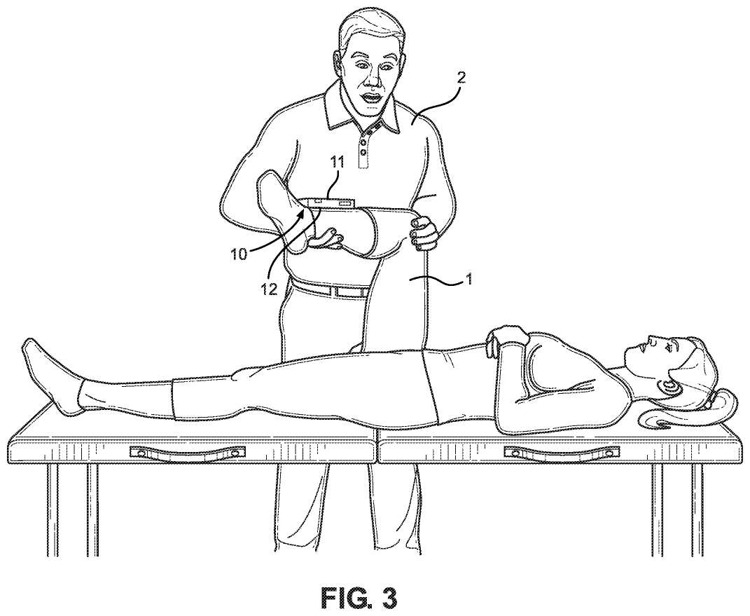

FIG. 3 depicts a side view of a physical therapist 2 utilizing substantially flat surface placement of base mount apparatus 10 on a patient's leg 1. As such, physical therapist places flat edge 15 of base mount 10 adjacently along patient's leg 1, or any other body part in need of measuring. Physical therapist 2 is then able to use HALO device 5 in conjunction with base mount 10 in order to take a proper and accurate measurement of patient's body 1 in order to determine patient's particular range of motion.

FIG. 4 depicts a perspective view of a physical therapist 2 utilizing substantially flat surface placement of base mount apparatus 10 on a patient's leg 1. As such, physical therapist places flat edge 15 of base mount 10 adjacently along patient's leg 1, or any other body part in need of measuring. Physical therapist 2 is then able to use HALO device 5 in conjunction with base mount 10 in order to take a proper and accurate measurement of patient's body 1 in order to determine patient's particular range of motion. Physical therapist 2 aligns the laser from HALO device 5 along crosshairs 28 of base mount 10 in order to increase accuracy of the measurement.

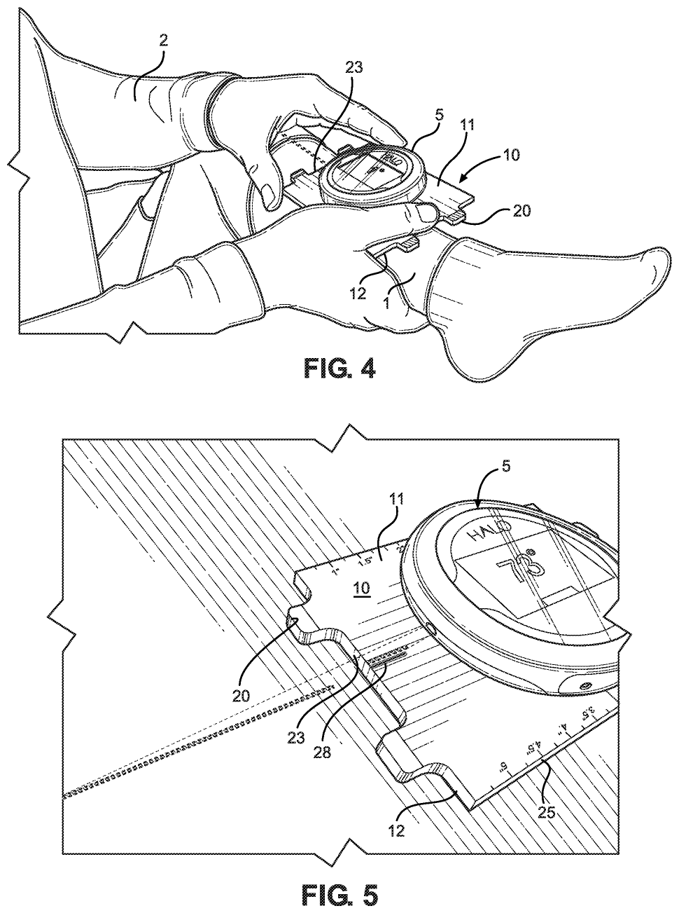

FIG. 5 depicts a perspective view of base mount 10 comprising HALO device 5 attachably connected to top surface 11 of base mount 10, wherein HALO device 5 has been laser aligned with crosshairs 28 of base mount 10. Laser alignment of HALO 5 with crosshairs 28 on top surface 11 of base mount 10 allows physical therapist 2 to obtain more accurate measurements using ruler 25.

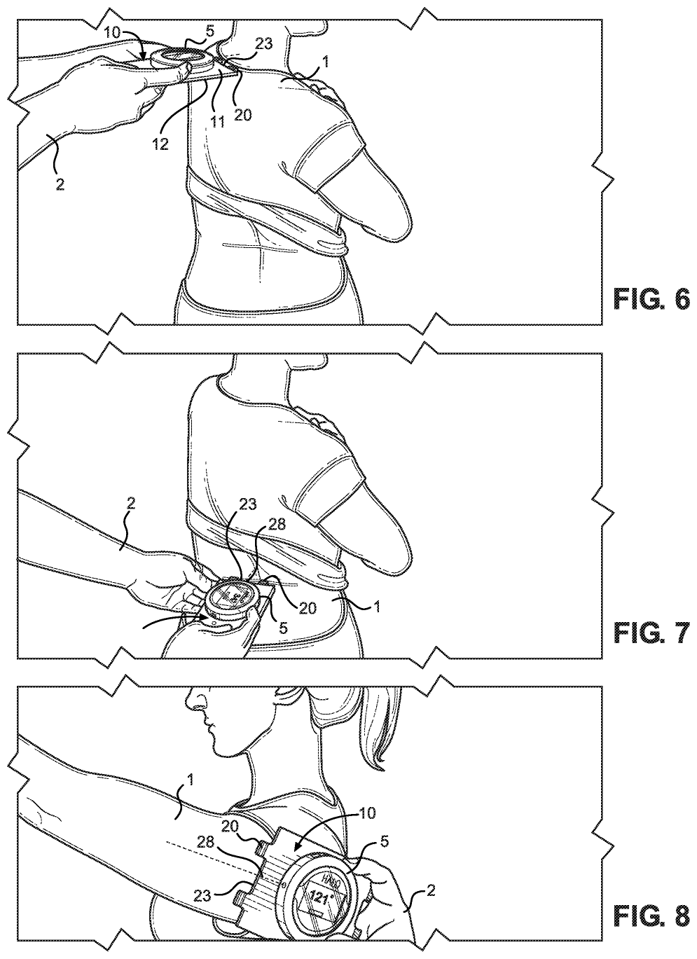

FIG. 6 depicts a side view of a physical therapist 2 utilizing prong 20 placement of base mount apparatus 10 on a patient's neck 1. FIG. 7 depicts a side view of a physical therapist 2 utilizing prong 20 placement of base mount apparatus 10 on a patient's back 1. FIG. 8 depicts a side view of physical therapist 2 utilizing prong 20 placement of base mount apparatus 10 on patient's arm 1. As such, physical therapist places a set of prongs 20 of base mount 10 adjacently along patient's body part 1 in need of measuring. Physical therapist 2 is then able to use HALO device 5 in conjunction with prongs 20 and prong spacing 23 of base mount 10 in order to take a proper and accurate measurement of patient's body 1, and thus, determine patient's particular range of motion. Physical therapist 2 aligns the laser from HALO device 5 along crosshairs 28 of base mount 10 in order to increase accuracy of the measurement with prongs 20 and prong spacing 23.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. A base mount apparatus for use in obtaining body measurements in conjunction with a range of motion measurement device, comprising:
a) a substantially planar body member having a top surface, a bottom surface, and a plurality of sides;
b) at least one set of alignment prongs having a first prong and a second prong; and
c) an attachment means, wherein said range of motion measurement device can be attachably mounted to said body member by way of said attachment means.

2. The base mount apparatus of claim 1, wherein said sides comprise at least one side having a substantially flat edge.

3. The base mount apparatus of claim 2, wherein said alignment prongs comprise a prong spacing located between said first prong and said second prong.

4. The base mount apparatus of claim 3, further comprising a plurality of crosshairs located on said top surface of said body member.

5. The base mount apparatus of claim 4, further comprising a ruler located on said top surface of said body member.

6. The base mount apparatus of claim 1, wherein said attachment means comprises a hook and loop pad.

7. The base mount apparatus of claim 1, wherein said attachment means comprises a snap in connection.

8. A method of obtaining range of motion body measurements comprising:
a) placing a base mount apparatus on a particular body part of a patient, wherein said base mount apparatus comprises:
i) a substantially planar body member having a top surface, a bottom surface, and a plurality of sides;
ii) at least one set of alignment prongs having a first prong and a second prong;
iii) an attachment means, wherein said range of motion measurement device can be attachably mounted to said body member by way of said attachment means;
b) aligning said range of motion measurement device with said base mount apparatus in order to receive a more accurate measurement; and
c) obtaining a range of motion measurement from said range of motion device.

9. The method of claim 8, wherein said sides comprise at least one side having a substantially flat edge.

10. The method of claim 9, wherein said alignment prongs comprise a prong spacing located between said first prong and said second prong.

11. The method of claim 10, further comprising a plurality of crosshairs located on said top surface of said body member.

12. The method of claim 11, further comprising a ruler located on said top surface of said body member.

13. The method of claim 8, wherein said attachment means comprises a hook and loop pad.

14. The method of claim 8, wherein said attachment means comprises a snap in connection.
